(19) United States
(12) Patent Application Publication
(10) Pub. No.: US 2002/0138864 A1
(43) Pub. Date: Sep. 26, 2002

(54) METHOD FOR TRANSFECTION OF AVIAN PRIMORDIAL GERM CELLS

(76) Inventors: JAE YONG HAN, SUWEON (KR); YEONG HO HONG, SUWEON (KR)

Correspondence Address: SAMUEL S. WOODLEY,PH.D., DARBY & DARBY P.C., 805 THIRD AVENUE, NEW YORK NEW YORK, NY, 10022, US

(21) Appl. No.: 09/183,729

(22) Filed: Oct. 30, 1998

(30) Foreign Application Priority Data
May 22, 1998 (KR) 18635/1998

(51) Int. Cl. A01K 67/027; C12N 5/06; C12N 15/87

(52) U.S. Cl. 800/21; 435/325; 435/440; 435/455; 800/24; 800/25

(57) ABSTRACT

The present invention relates to a method for transfection of avian primordial germ cells which comprises a step of electroporating a foreign gene into avian primordial germ cells after addition of dimethylsulfoxide( “DMSO”) to culture medium containing the cells, and a method for production of transgenic Aves by employing the avian primordial germ cells transfected thereby. The DMSO-electroporation method of the invention has various advantages over the conventional transfection methods in light of high transfection efficiency, safety, and simple manipulation. Accordingly, a large number of primordial germ cells transfected with a gene of interest can be efficiently produced by the invention, which, in turn, may increase the rate of transfer of exogenous DNA into recipient embryos.

### FIELD OF THE INVENTION

The present invention relates to a method for transfection of avian primordial germ cells and its use, more specifically, to a method for transfection of avian primordial germ cells which comprises a step of electroporating a foreign gene into avian primordial germ cells after addition of dimethylsulfoxide to culture medium containing the cells, and a method for production of transgenic Aves by employing the avian primordial germ cells transfected thereby.

### BACKGROUND OF THE INVENTION

During the last 10 years, many studies on the production of transgenic Aves have been actively carried out in the art(see: Bosselman, R. A. et al., Science, 243:533-535, 1989; Perry, M. M. and Sang, H. M., Transgenic Res., 2:125-133, 1993; Love, J. et al., Biotech., 12:60-63, 1994), and several methods for manipulating avian genome using primordial germ cells(hereinafter referred to as “PGCs”) or blastodermal cells are currently in development. The PGCs are highly attractive since during embryogenic development, avian germ cells provide various time points useful for manipulation.

In chick embryos, PGCs appear to segregate from the somatic cells as early as stage X(see: Eyal-Giladi, H. and Kochav, S., Dev. Biol., 49:321-337, 1976), while expressing characteristic carbohydrate epitopes(see: Karagenc, L. et al., Dev. Gen., 19:290-301, 1996). The stage X embryo has been used universally to produce somatic and germline chimeras(see: Thoraval, P., Poult. Sci., 73:1897-1905, 1994). As primitive streak develops, PGCs can be found in a crescent-shaped region at area pellucida and area opaca, which is known as germinal crescent. Further, as blood islands and the associated extra- and intra-embryonic vasculature develop, the PGCs actively migrate into circulatory system and are found in blood around stage 15 (see: Hamburger, V. and Hamilton, H. L., J. Morphol., 88:49-92, 1951). After this passive migration, the PGCs actively migrate from the blood along the dorsal mesentery until they take up their residence in the germinal ridge.

Because of this unique and well-defined migration, germ cells which were isolated from the embryonic gonad, the embryonic blood and the germinal crescent have been used to generate germ line chimeras(see: Simkiss, K., Protoplasma, 151:164-166, 1989; Ohno, T. et al., Exp. Anim., 45:347-352, 1996). For example, PGCs from embryonic gonads were successfully cultured in vitro for 5 days(see: Chang, I. K. et al., Cell Biol. Int., 19:143-149, 1995) and transfected PGCs were found in the recipient's gonad after injection into recipient embryos(see: Savva, D. et al., Research in Veterinary Science, 50:131-133, 1991).

However, despite of potential usefulness of the PGCs, transfection efficiency of the conventional methods is very low, which restricts production of transgenic Aves. Up to date, the conventional methods for transfecting PGCs have been limited to liposome-mediated method, retroviral infection, and microinjection(see: Allioli, N. et al., Dev. Biol., 165:30-37, 1994; Li, Y. et al., Transgenic Res., 4:26-29, 1995; Watanabe, M. et al., Mol. Reprod. And Dev., 38:268-274, 1994). However, these methods have revealed several shortcomings in light of transfection efficiency, safety, and complexities of manipulation as follows: Generally, the transfection efficiency of liposome-mediated method in avian PGCs ranges only 1.5% to 26% (see: Han, J. Y. et al., AJAS, 7:427-434, 1994; Wentworth, B. et al., Beltsville Symposium in Agriculture Research, pp.202-227, 1996). On the other hand, retroviral vectors have raised a problem of poor public acceptance with regard to safety although they have been successful in stably incorporating exogenous DNA into the genomic DNA(see: Bosselman, R. A. et al., Science, 243:533-535, 1989; Vick, L. et al., Proc. Royal Soc. London(B), 251:179-182, 1993). Microinjection of DNA into a newly fertilized ovum is not appropriate for the industrial production of transgenic Aves since it requires very complicated manipulation(see: Love, J. et al., Biotech., 12:60-63, 1994).

Under the circumstances, there has been strong reason for developing an alternative method for efficient transfection of avian PGCs.

### SUMMARY OF THE INVENTION

The present inventors have made an effort to solve the problems of the conventional transfection methods, and finally developed a novel method for transfection of avian PGCs which has been dramatically ameliorated in light of high transfection efficiency, safety, and simple manipulation, based on a finding that: the transfection efficiency in avian PGCs can be highly improved by establishing optimal conditions for electroporation and adding dimethylsulfoxide to culture medium containing PGCs at an adequate concentration.

A primary object of the invention is, therefore, to provide a method for transfection of avian primordial germ cells with a foreign gene.

The other object of the invention is, to provide a method for production of transgenic Aves by employing the avian primordial germ cells transfected by the said method.

### BRIEF DESCRIPTION OF DRAWINGS

The above and the other objects and features of the present invention will become apparent from the following description given in conjunction with the accompanying drawings, in which:

FIG. 1 is a graph showing transfection efficiencies for gPGCs depending on electroporation conditions.

FIG. 2A is a photograph showing a result of X-gal staining of gPGCs which were transfected with lacZ using liposomes and cultured for 24 hrs.

FIG. 2B is a photograph showing a result of X-gal staining of gPGCs which were electroporated with lacZ in the presence of DMSO and cultured for 24 hrs.

FIG. 3 is a photograph showing a result of PCR analysis of DNA from the gonads, liver and heart of hatched chicks transferred with transfected gPGCs.

### DETAILED DESCRIPTION OF THE INVENTION

The present inventors employed the technique of electroporation which is a well known method for transfection of eukaryotic cells in the transfection of avian primordial germ cells. To establish the optimal electroporation conditions for avian primordial germ cells and fibroblasts, a series of electroporations were carried out, respectively, in chick embryonic gonadal cells comprising gonadal primordial germ cells(hereinafter referred to as “gPGCs”) and stroma cells and in chick embryonic fibroblasts(hereinafter referred to as “CEFs”), which were isolated from 6-day-old embryos of White Leghorn. In this connection, pCMV β vector containing lacZ gene was employed as a reporter gene. gPGCs were isolated from the transfected gonadal cells, and transfection efficiencies were measured in the gPGCs and CEFs by X-gal staining. As a result, it was found that: electroporation for chick gPGCs is preferably carried out under a condition of 230 to 270V, 900 to 950 μF, most preferably 250V, 950 μF; and, electroporation for CEFs is preferably carried out under a condition of 230 to 270V, 250 to 350 μF, most preferably 250V, 300 μF.

Further, to investigate the effect of dimethylsulfoxide(DMSO) on the transfection efficiency of electroporation, electroporations were carried out in chick gPGCs and CEFs in the presence or absence of DMSO under the most preferable electroporation conditions described above. And then, β-galactosidase activities in cytosolic extracts prepared from the transfected gPGCs and CEFs, respectively, were assayed, which revealed that: addition of DMSO to the culture medium preferably at a concentration of 1 to 1.5% (v/v), most preferably 1.25% (v/v) can highly increase the transfection efficiency in gPGCs in cell type-specific manner. In describing the transfection method of the present invention, “DMSO-electroporation” is employed to mean the method for transfection of PGCs by electroporating a foreign gene into PGCs after addition of DMSO.

In addition, transfer of the gPGCs which were transfected with a foreign gene by DMSO-electroporation into a recipient chick embryo may provide a potential method for production of transgenic chicken which expresses the foreign gene in its gonads.

In a preferred embodiment of the present invention, gPGCs isolated from an embryo of White Leghorn was preferably used as the avian PGCs, where the Aves includes chickens, quails, pheasants, turkeys and ducks, and the PGCs can be prepared from embryonic gonad, embryonic blood or germinal crescent.

The present invention is further illustrated in the following examples, which should not be taken to limit the scope of the invention.

### EXAMPLE 1

### Preparation and Culture of Gonadal Primordial Germ Cells and Embryonic Fibroblasts

Fertilized eggs from White Leghorn stock were incubated in a humidified, still-air incubator at 37° C. for 6 days. Gonadal cells comprising gonadal primordial germ cells(gPGCs) and stroma cells were prepared from gonads which were isolated from the 6-day-old embryos, by mechanically dispersing after treatment of 0.25% trypsin-0.02% EDTA(Sigma, USA) for 10 min. On the other hand, chick embryonic fibroblasts(CEFs) were prepared from the minced skin of the 6-day-old embryos as described above. The CEFs thus prepared were cultured in Dulbecco's modified Eagle's medium(DMEM, Gibco BRL, USA) supplemented with 10% fetal bovine serum(FBS), 100 units/ml penicillin and 100 μg/ml streptomycin for 12 hours. And then, the CEFs were subcultured for transfection.

### EXAMPLE 2

### Optimization of Electroporation Conditions

### EXAMPLE 2-1

### Transfection of gPGCs and CEFs by Electroporation

To establish the optimal conditions for electroporation of gPGCs and CEFs, a series of electroporations were carried out, while varying voltage and capacitance of electric pulse in a range of 200 to 300V and 250 to 950 μF. In this connection, pCMV β (Clontech, USA) including bacterial lac Z gene was employed as a reporter gene for convenient measurement of transfection efficiency. The pCMV β plasmid was prepared using large-scale matrix-based purification kit(Quiagen, Inc., USA) according to manufacturer's instructions. 1.6×10⁵ gonadal cells and 2.3×10⁶ CEFs prepared in Example 1 were mixed with 20 μg of plasmid DNA in 400 μl of Opti-MEM(Gibco BRL, USA), respectively, and let to stand for 1 min at room temperature. And then, electroporations were carried out under the said conditions. After the electroporation, the gonadal cells were cultured in DMEM supplemented with 10% FBS, 10 ng/ml insulin-like growth factor-1 (IGF-1, Sigma, USA), 10 ng/ml basic fibroblast growth factor(bFGF, Sigma, USA) and 10 units/ml murine leukemia inhibitory factor(murine LIF, Sigma, USA) for 24 hours, while CEFs were cultured in DMEM supplemented with 10% FBS, 100 units/ml penicillin and 100 gμ/ml streptomycin for 24 hours.

### EXAMPLE 2-2

### Measurement of Transfection Efficiency by X-gal Staining

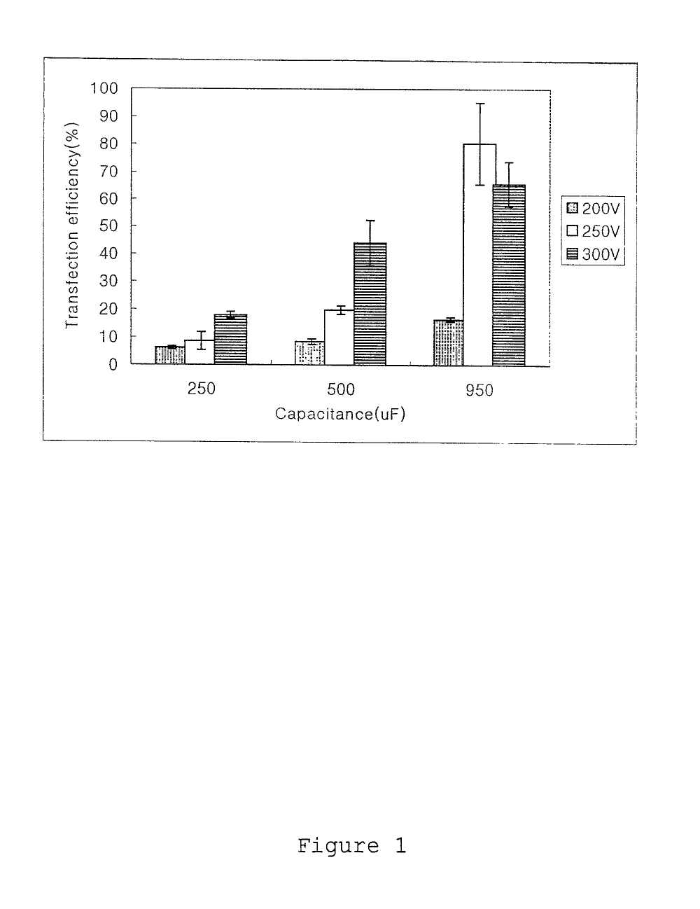

The transfected gPGCs were purified from the transfected gonadal cells by ficoll density gradient centrifugation(see: Chang, I. K. et al., Cell Biol. Int. Rep., 16:853-857, 1992) and identified by PGC-specific PAS(periodic acid-schiff) staining, which indicated that the purity of the gPGCs is about 80%. The purified gPGCs and transfected CEFs prepared in Example 2-1 were washed with phosphate-buffered saline(PBS), fixed in 1% glutaraldehyde in PBS for 5 minutes, washed twice with PBS, and stained with X-gal. Transfection efficiencies in gPGCs and CEFs were measured as percentage of stained cells in the total cells. The results revealed that an optimal pulse can be achieved with 250V, 950 μF for gPGCs(see: FIG. 1) and 250V, 300 μF for CEFs, respectively.

### Example 3

### Effect of DMSO on Transfection Efficiency of Electroporation

Efficiencies of transfection by electroporation in gPGCs and CEFs were tested with or without DMSO by measuring β-galgactosidase activities as followings: Gonadal cells and CEFs were electroporated under a condition of 250V, 950 μF and 250V, 300 μF, respectively, with or without DMSO(1.25%, v/v) analogously as in Example 2-1 and cultured for 24 hours, and gPGCs were purified from the gonadal cells analogously as in Example 2-2. And then, the gPGCs and CEFs were lysed in 100 μl of lysis buffer(0.2% triton X-100, 100 mM potassium phosphate buffer, pH 7.8 , 1 nM dithiothreitol) to obtain cytosolic extracts. The 2-galactosidase activities in the cytosolic extracts were assayed according to Sambrook et al. (see: Sambrook, J. et al., Molecular Cloning: A Laboratory Manual, 2 nd ed., Cold Spring Harbor, N.Y., USA: Cold Spring Harbor Lab. Press, vol(3), pp. 66-67), and summarized in Table 1 below. At this time, protein concentrations of the extracts were determined by Bradford method using bovine serum albumin as a standard(see: Bradford, M. M., Anal. Biochem., 7:248-254, 1976). As can be seen in Table 1 below, the β-galactosidase activity in cytosolic extract of gPGCs was increased by 2.2-fold upon addition of DMSO. On the other hand, no significant difference was found in CEFs upon addition of DMSO.

TABLE 1
Effect of DMSO on transfection efficiency
(unit: β-galactosidase activity/mg protein)

Cell Types

| | gPGCs | CEFs |
|---|---|---|
| Control | 0.243 ± 0.01 | 0.353 ± 0.04 |
| DMSO-treatment | 0.538 ± 0.18 | 0.440 ± 0.06 |

These results strongly suggest that the role of DMSO may be related to increase of permeability of cell membrane or stabilization of the membrane after electric pulse, and it may depend on the cell type.

### EXAMPLE 4

### Comparison of Transfection Efficiencies of DMSO-electroporation and Liposome-mediated Transfection in gPGCs

Gonadal cells were prepared from gonads as analogously in Example 1 and cultured in DMEM supplemented with 10% FBS, 10 ng/ml IGF-1, 10 ng/ml bFGF and 10 units/ml murine LIF in 96-well plate for 24 hours. And then, transfection was carried out by employing lipofectAMINE(Gibco BRL, USA) according to the manufacturer's instructions: First, 400 ng of pCMV β diluted in 20 μl of Opti-MEM was added to 4.8 μg of lipofectAMINE diluted with 20 μl of Opti-MEM. After mixing carefully, the mixture was allowed to form a liposome:DNA complex at room temperature for 30 min. The gonadal cells were washed once with PBS to remove serum, received the liposome:DNA complex, and incubated for 3 hours at 37° C.

On the other hand, electroporation of gonadal cells was carried out analogously as in Example 3 in the presence of 1.25% (v/v) DMSO. The transfected cells were cultured in DMEM supplemented with 10% FBS, 10 ng/ml IGF-1, 10 ng/ml bFGF and 10 units/ml murine LIF for 24 hours. And then, gPGCs were purified from the gonadal cells analogously as in Example 2-2.

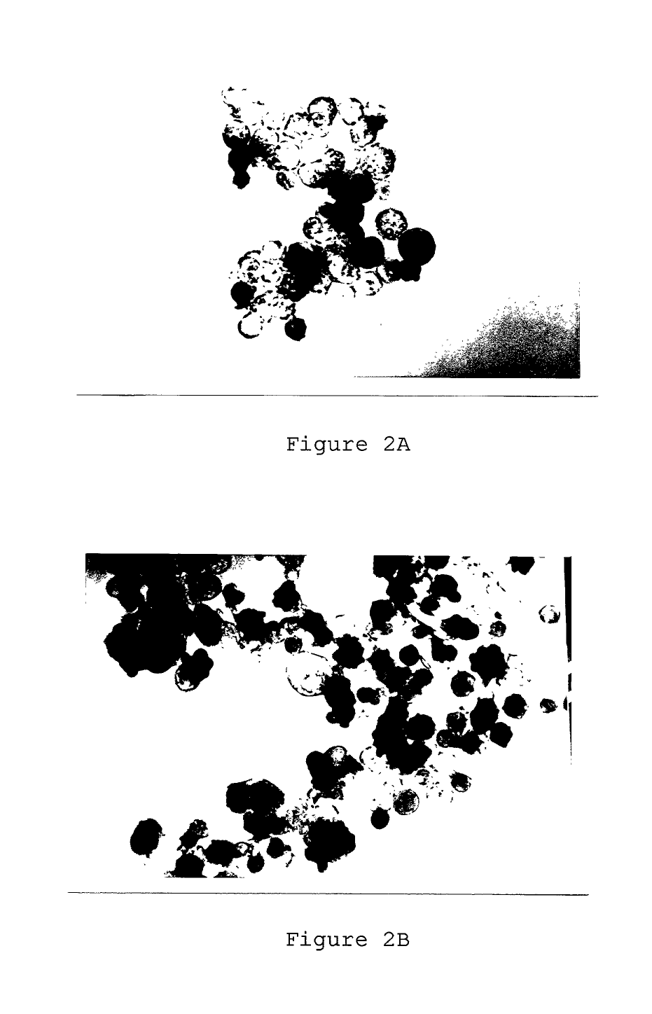

The transfection efficiencies of the two methods, i.e., liposome-mediated transfection and DMSO-electroporation of the present invention were measured by X-gal staining(see: FIGS. 2A and 2B), whose results were summarized in Table 2 below. As can be seen in Table 2, transfection efficiency of liposome was 17.5±1.8 %, while that of electroporation was 80.3±14.8% which is 4.6-fold higher than that of liposome.

TABLE 2
Transfection efficiencies of liposome and DMSO-electroporation

| Transfection Method | Transfection Efficiency (%) |
|---|---|
| LipofectAMINE | 17.5 ± 1.8 |
| Electroporation | 80.3 ± 14.8 |

### EXAMPLE 5

### Injection of gPGCs into Recipient Embryos and PCR Analysis

Approximately 200 gPGCs transfected with pCMVβ by DMSO-electroportion under a condition of 250V, 950 μF were injected into the dorsal aorta of 2.5-day-old(stage 17) chick embryos through access window. The access window was sealed with paraffin sealing film and the eggs were incubated for an additional 3.5 days(i.e., until stage 29) 7.5 days(i.e., until stage 36) or until hatching in the hatchery. Then, template DNAs were extracted using proteinase K from gonads of the embryos of stage 29 and stage 36, and from gonads, liver, and heart of the hatched chicks, respectively, according to Clinton's method(see: Clinton, M., Animal Gen., 25:361-362, 1994), and subjected to PCR analyses with the following lacZ gene-specific primers:

| | |
|---|---|
| 5′- AGA TGC ACG GTT ACG ATG C -3′ | (SEQ ID |
| | NO: 1) |
| 5′- GGT CAA ATT CAG ACG GCA AAC G -3′ | (SEQ ID |
| | NO: 2) |

At this time, genomic DNA from gonads of uninjected embryos was used as a negative control. PCR amplification products were resolved on a 2% (w/v) agarose gel. As a result, it was found that the rate of detecting lac Z gene in the gonads was 100% and 67% in 6-day-old (stage 29) and 10-day-old (stage 36) recipient embryos, respectively, and 41% in the hatched chicks, which was summarized in Table 3 below.

TABLE 3
Number of embryos containing
exogenous lac Z gene in gonads after injection

| | | Number of Embryos |
|---|---|---|
| | | Containing Exogenous |
| Stage of Embryos | Number of | lac Z gene in |
| (incubation days) | Embryos Injected | Gonads (%) |
| Stage 29 (6 days) | 9 | 9 (100%) |
| Stage 36 (10 days) | 6 | 4 (67%) |
| Hatched Chicks | 27 | 11 (41%) |
| (21 days) | | |

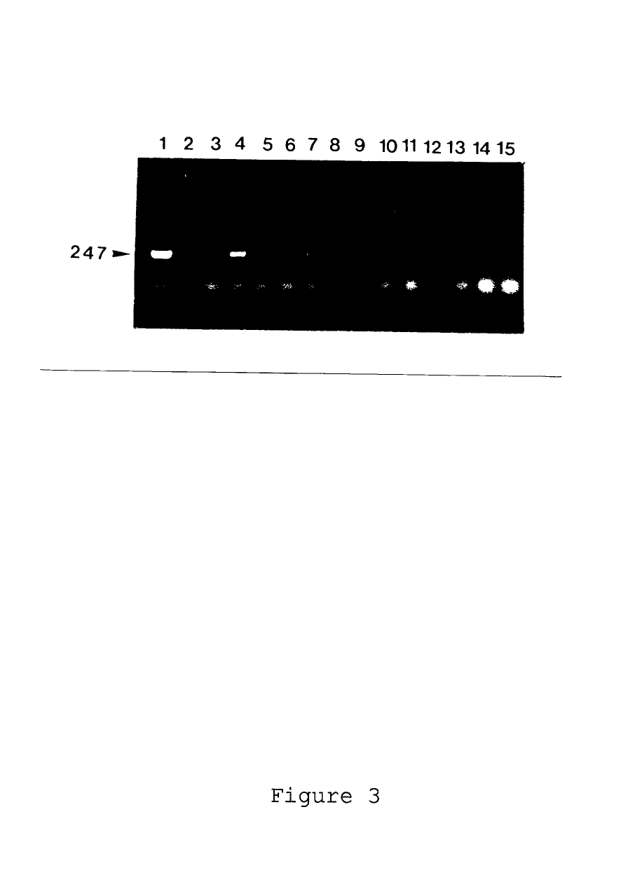

On the other hand, FIG. 3 shows the result of PCR analyses of DNAs from the gonads, liver, and heart of the hatched chicks transferred with the transfected gPGCs. The exogenous lac Z gene was detectable only in the gonad and not in the liver and heart (see: FIG. 3). In FIG. 3, lane 1 represents 247 bp lac Z gene fragment; lane 2, chicken genomic DNA; lane 3, negative control; lanes 4, 7, 10 and 13, gonadal DNA; lanes 5, 8, 11 and 14, liver DNA; and, lanes 6, 9, 12 and 15, heart DNA.

As clearly illustrated and demonstrated as aboves, the present invention provides a method for transfection of avian primordial germ cells with foreign gene and a method for production of transgenic Aves by using the avian primordial germ cells transfected thereby. In accordance with the present invention, transfection efficiency in PGCs can be highly improved by adding DMSO into culture medium containing PGCs and electroporating foreign DNA under optimal conditions. The DMSO-electroporation method has various advantages over the conventional transfection methods in light of high transfection efficiency, safety, and simple manipulation. Accordingly, a large number of primordial germ cells transfected with a gene of interest can be efficiently produced by the invention, which, in turn, may increase the rate of transfer of exogenous DNA into recipient embryos.

What is claimed is:

1. A method for tranfection of avian primordial germ cell which comprise a step of electroporating a foreign gene into avian primordial germ cells after addition of dimethylsulfoxide to culture medium containing the cells.

2. The method of claim 1, wherein the Aves are chickens, quails, pheasants, turkeys or ducks.

3. The method of claim 1, wherein the primordial germ cells are prepared from embryonic gonad, embryonic blood or germinal crescent.

4. The method of claim 1, wherein the dimethylsulfoxide is added to the culture medium at a concentration of 1 to 1.5% (v/v).

5. The method of claim 1, wherein the electroporation is carried out under a condition of 230 to 270V, 900 to 950 μF.

6. A method for production of transgenic Aves which comprises the steps of:
injecting avian primordial germ cells transfected with a foreign gene by the method of claim 1, into dorsal aorta of an avian embryo; and, culturing the embryo until hatching.


SEQUENCE LISTING (2 sequences)

SEQ ID NO: 1 - DNA, 19 bp, Artificial Sequence
  Description of Artificial Sequenceprimer
agatgcacggttacgatgc

SEQ ID NO: 2 - DNA, 22 bp, Artificial Sequence
  Description of Artificial Sequenceprimer
ggtcaaattcagacggcaaacg